(19) United States
(12) Patent Application Publication
(10) Pub. No.: US 2003/0059453 A1
(43) Pub. Date: Mar. 27, 2003

(54) Kit for mixing substances for the creation of cosmetic products

(76) Inventor: Maria Prada Bianchi, Milano (IT)

Correspondence Address: SHERIDAN ROSS PC, 1560 BROADWAY, SUITE 1200, DENVER, CO, 80202

(21) Appl. No.: 10/253,566

(22) Filed: Sep. 23, 2002

(30) Foreign Application Priority Data
Sep. 25, 2001 (IT) MI2001A 001989

(51) Int. Cl. A61K 9/00; B65D 69/00; B65D 71/00

(52) U.S. Cl. 424/401; 206/581

(57) ABSTRACT

A kit for mixing substances for the creation of cosmetic products comprises: a first member containing therewithin a first substance and being closed at one end by a breakable cover; a second member containing therewithin a second substance and being closed at one end by a breakable cover; and an intermediate member able to receive in a relationship of engagement the ends provided with the breakable cover of the first and second members, said intermediate member comprising cutting means able to tear said breakable covers of the first and second members to allow discharge of the respective substances and defining a mixing chamber able to allow the passage of said substances for mixing thereof so as to obtain a ready-to use cosmetic product.

### DESCRIPTION

The present invention refers to a kit for mixing substances for the creation of cosmetic and/or dermatological products, in particular for instantaneous preparation of an 1-ascorbic acid solution for cosmetic use as a stimulator of collagen production.

Solutions based on 1-ascorbic acid are generally obtained by mixing a powder product containing active principles with a liquid product or serum.

The powder product containing 1-ascorbic acid active principles is generally packaged separately from the serum and is mixed with the serum only immediately before use.

Different types of containers for ascorbic acid compositions are known on the market.

U.S. Pat. No. 6,010,760 in the name of L'Oréal discloses a container formed by two chambers separated from each other by a partition. The fist chamber contains an ascorbic acid powder and the second chamber contains a hydrophilic carrier. A mixing mechanism is provided in the form of a stopper, able to rupture the partition between the two chambers so that the powder is mixed with the liquid inside the container.

Said container proves structurally complex and not very versatile.

The object of the present invention is to provide a kit for mixing substances for the creation of cosmetic products that is able to mix two substances previously kept separate, prior to use.

Another object of the present invention is to provide such a kit for mixing substances that is practical and simple for the user to use.

Yet another object of the present invention is to provide such a kit for mixing substances that is versatile and able to mix different substances for the creation cosmetic products that are different in type and quantity.

Yet another object of the present invention is to provide such a kit for mixing substances that is inexpensive and simple to make.

These objects are achieved in accordance with the invention with the characteristics listed in appended independent claim 1.

Advantageous embodiments of the invention are apparent from the dependent claims.

The kit for mixing substances for creating cosmetic products according to the invention comprises three separate members. A first member contains on its inside a fist substance and is closed at one end thereof by a breakable cover. The second member contains on its inside a second substance and is closed at one end thereof by a breakable cover. An intermediate member is able to receive engagingly the ends provided with a breakable cover of the first and second members.

The intermediate member comprises cutting means able to tear the breakable covers of the first and second members to allow discharge of the respective substances. Within the intermediate member a mixing chamber is defined, able to allow the passage of said substances for mixing thereof so as to obtain a ready-to-use cosmetic product.

In this manner the two different substances are kept separate in the first and second members. When the first and second members are coupled to the intermediate member, the covers of the first and second members are ruptured and the two substances are mixed together giving rise in particular to a cosmetic product.

The kit according to the invention affords the advantages of having a simple structure and being extremely versatile by virtue of the interchangeability of the members that compose it.

Further characteristics of the invention will be made clearer by the detailed description that follows, referring to a purely exemplary and therefore non-limiting embodiment thereof, illustrated in the appended drawings, wherein:

FIG. 1 is an exploded plan view illustrating the kit for mixing substances to create cosmetic products according to the invention;

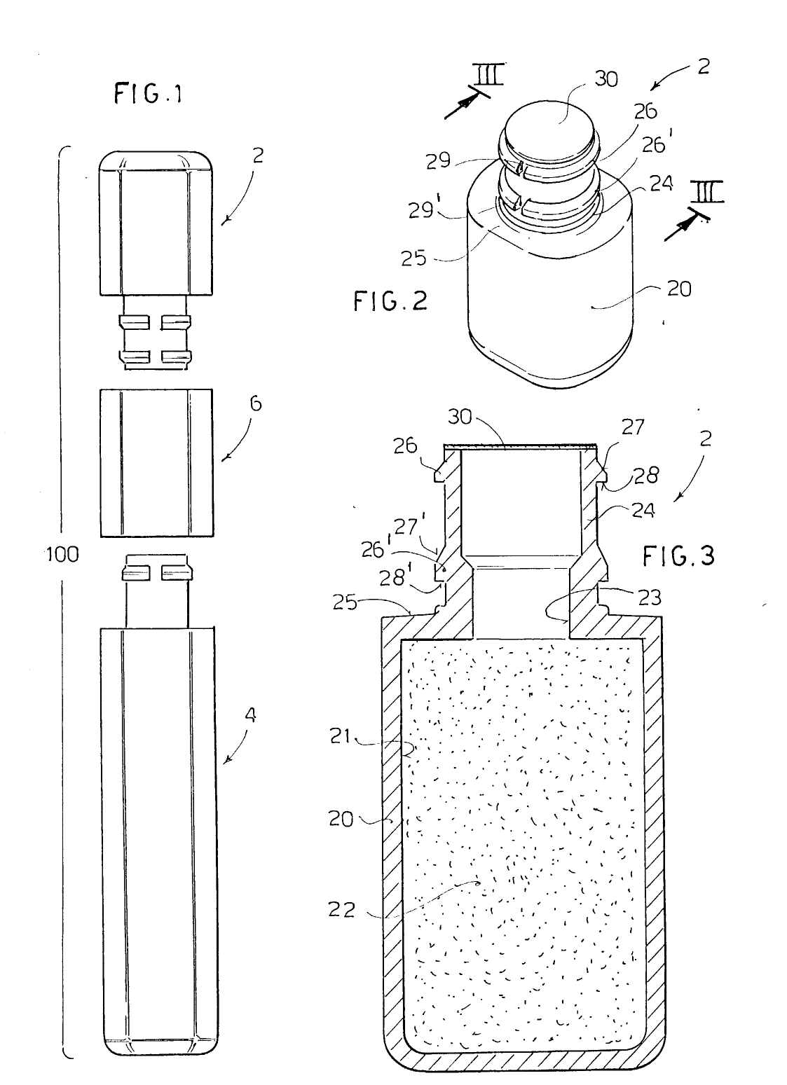

FIG. 2 is a perspective view illustrating a first end member of the kit of FIG. 1;

FIG. 3 is an axial sectional view along the plane of section III-III of FIG. 2;

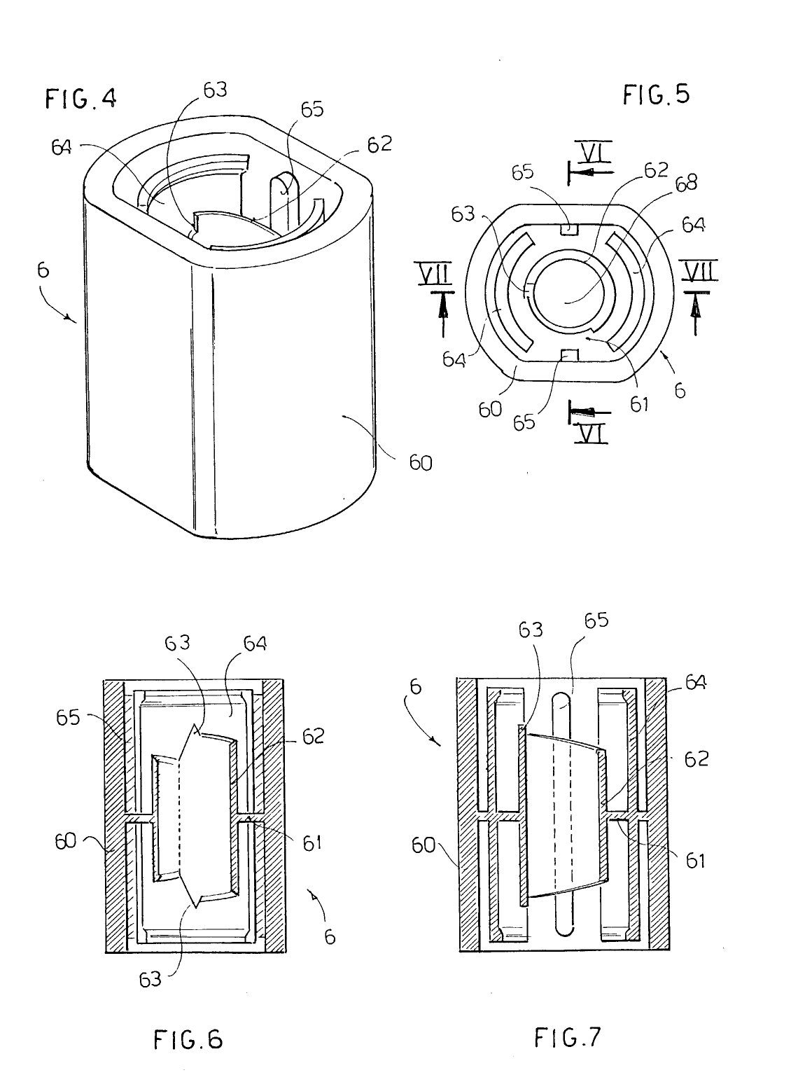

FIG. 4 is a perspective view illustrating an intermediate member of the kit of FIG. 1;

FIG. 5 is a top plan view of the intermediate member of FIG. 4;

FIG. 6 is an axial sectional view along the plane of section VI-VI of FIG. 5;

FIG. 7 is an axial sectional view along the plane of section VII-VII of FIG. 5;

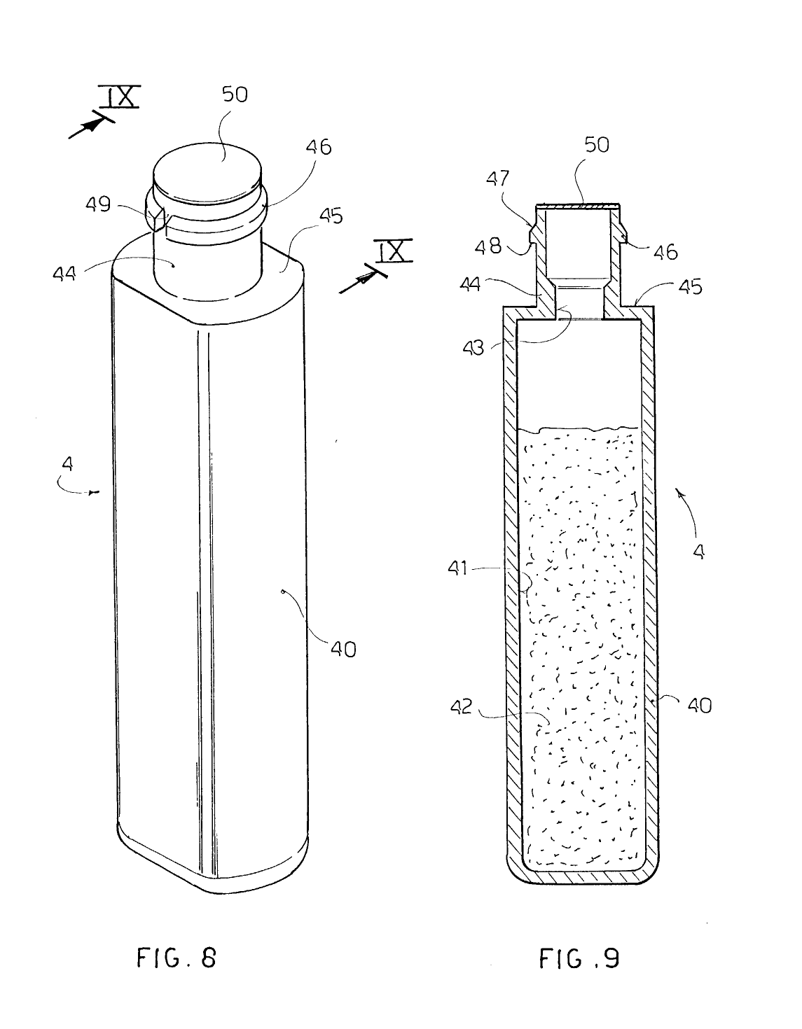

FIG. 8 is a perspective view of a second end member of the kit of FIG. 1;

FIG. 9 is an axial sectional view along the sectional plane IX-IX of FIG. 8;

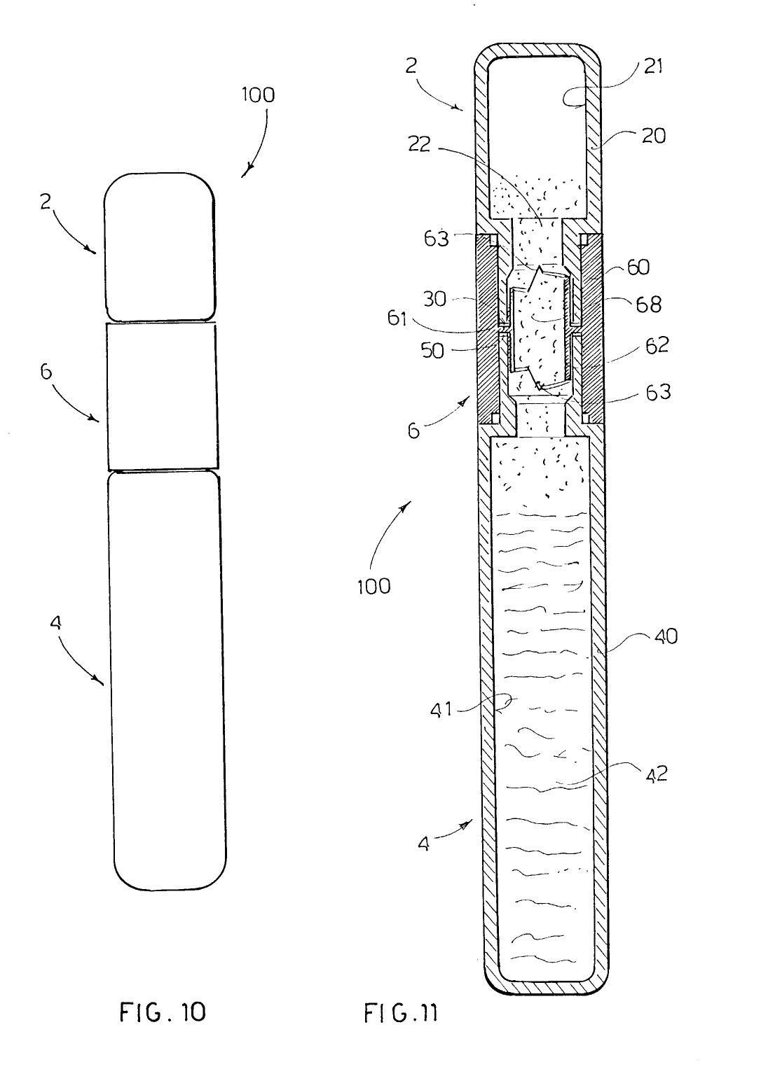

FIG. 10 is a plan view of the kit according to the invention, shown assembled;

FIG. 11 is an axial sectional view of the kit according to the invention assembled, raised and in a mixing stage.

The kit for mixing of substances for the creation of cosmetic products according to the invention is described with the aid of the figures.

FIG. 1 shows a kit for mixing substances for the creation of cosmetic products, according to the invention, designated as a whole with reference numeral 100.

The kit 100 comprises a first end member 2, a second end member 4 and an intermediate member 6 comprised between the first end member 2 and the second end member 4.

As shown in FIGS. 2 and 3, the first end member consists of a container comprising a substantially cylindrical body 20, hollow on the inside, such as to define a cavity 21 wherein a substance 22 for cosmetic use is contained.

The substance 22 is preferably a powder product containing active principles of 1-ascorbic acid.

The upper wall of the body 20 has an axial hole 23 around which a neck 24 rises, protruding axially outward. The neck 24 has a smaller diameter than the body 20 so as to give rise to a shoulder 25 on the upper surface of the body 20.

Two annular collars 26, 26′, protruding outward, parallel and spaced apart from each other, are disposed on the neck 24. Each collar 26, 26′ has an oblique upper surface 27, 27′ with respect to the side wall of the neck 24 and a bottom surface 28, 28′ protruding radially form the side wall of the neck 24.

Each collar 26, 26′ has a pair of notches 29, 29′ disposed in diametrically opposite positions (in FIG. 2 only one notch is visible for each collar). Each notch 29 of the top collar 26 is in register with the respective notch 29′ of the bottom collar 26′, according to a vertical straight line parallel to the axis of the neck 24.

A breakable film 30 is fixed on the upper rim of the neck 24. The film 30 acts as a closure and prevents the powder 22 from escaping from the first end member 2.

The second end member 4 is described with reference to FIGS. 8 and 9.

The second end member 4 consists of a container comprising a substantially cylindrical body 40, hollow on the inside, such as to define a cavity 41 wherein a substance 42 for cosmetic use is contained. The body 40 of the second member has substantially the same diameter as the body 20 of the first member and a greater height than the first member.

The substance 42 is preferably a liquid product, such as, for example, a serum able to dissolve the powder product 22 contained in the first member.

The top wall of the body 40 has an axial hole 43 around which a neck 44 rises, protruding axially outward. The neck 44 has a smaller diameter than the body 40 so as to give rise to a shoulder 45 on the upper surface of the body 40.

An outwardly protruding annular collar 46 is disposed on the neck 44. The collar 46 has an oblique upper surface 47 with respect to the side wall of the neck 44 and a bottom surface 48 protruding radially from the side wall of the neck 44.

The collar 46 has a pair of notches 49 (only one visible in FIG. 8) disposed in diametrically opposite positions.

A breakable film 50 is fixed on to the upper edge of the neck 44. The film acts as a closure and prevents the liquid product 42 from escaping from the second end member 4.

The intermediate member 6 is described with the aid of FIGS. 4-7. The intermediate member 6 comprises a substantially cylindrical body 60, hollow on the inside, having substantially the same transverse measurements as those of the bodies 20 and 40 of the first end member 2 and the second end member 4.

A transverse bulkhead 61, which divides the inside of the body 60 into two perfectly symmetrical chambers, is provided in a median position within the body 60. The transverse bulkhead 61 supports a cylinder 62, hollow on the inside, disposed in an axial position and defining on its inside a mixing chamber 68.

The height of the cylinder 62 is slightly less than the height of the body 60. The thickness of the wall of the cylinder 62 is extremely small and the cylinder 62 has in its top and bottom edges a cusp 63 acting as a blade and able to tear the breakable films 30 and 50 of the first end member 2 and the second end member 4.

Two longitudinal tongues 64 disposed in diametrically opposite positions, having a length substantially equal to that of the body 60, are provided between the inner wall of the body 60 and the outer wall of the cylinder 62. Each tongue 64 has two parts which protrude axially below and above the transverse bulkhead 61, so as to be able to bend radially.

Each tongue 64 has a substantially curved cross section, shaped like a quarter of a circle, defining a slightly larger circumference than that defined by the outer surface of the necks 24 and 44 of the first and second member. In this manner, an annular gape or space having a width substantially equal to the thickness of the necks 24 and 44 of the first and second members, inclusive of the respective collars 26, 26′ and 46, is defined between the inner surface of the tongues 64 and the outer surface of the cylinder 62.

Two longitudinal projections or protrusions 65, disposed in diametrically opposite positions and alternating exactly with the longitudinal tongues 64 are provided in the inner wall of the body 60. The longitudinal protrusions 65 are of substantially the same length as the body 60 and are of such a size as to be able to slide in the recesses 29, 29′ of the collars 26, 26′ of the first member and in the recesses 49 of the collar 46 of the second member.

Operation of the kit 100 will now be described. As shown in FIGS. 10 and 11, the kit 100 is assembled engaging the necks 24 and 44 of the first end member 2 and the second end member 4 in the respective end openings of the intermediate member 6.

To be precise, the neck 24 of the first member 2 enters the space between the inner surface of the tongues 64 of the intermediate member 6 and the outer surface of the cylinder 62 of the intermediate member and the longitudinal projections 65 of the intermediate member enter the slots 29, 29′ of the collars 26, 26′. The longitudinal projections 65 thus act as guides and allow correct insertion of the neck 24 inside the intermediate member 6.

The collars 26, 26′ of the neck 24 push the longitudinal tongues 64 of the intermediate member, which bend outward and at the same time exert a grip on the collars 26, 26′ of the neck 24. As the neck 24 is inserted in the intermediate member 6, the cusp-shaped blade 63 of the cylinder 62 of the intermediate member penetrates the covering film 30 of the first member, tearing it. At this point, since the film 30 is torn, the powder 22 contained inside the first member can escape from the neck 24 and flow through the mixing chamber 68 of the cylinder 62.

In the same manner the neck 44 of the second member engages in the intermediate member 6 and the cusp-shaped point 63 of the cylinder 62 of the intermediate member tears the covering film 50 of the second member. In this manner the powder product 22 enters the cavity 41 of the second member through the mixing chamber 68 of the cylinder 62 and mixes with the liquid 42.

To aid mixing, the assembled kit 100 is shaken by the user and the liquid 42 with the powder 22 are mixed, passing alternately through the mixing chamber 68 of the cylinder 62 of the intermediate member.

Once mixing has been completed, the liquid containing the dissolved active principles is deposited in the cavity 41 of the second container 4. At this point, the intermediate member 6 with the first member 2 assembled, is disengaged from the neck 44 of the second member 4, where the liquid remains, mixed with the powder, ready to be used for cosmetic purposes.

In this description an embodiment has been described that provides three separate members 2, 6 and 4, which are assembled with one another before use. However, the three members 2, 6 and 4 cam be provided already assembled and in this case engagement between the neck 24 of the first member and the intermediate member and engagement between the neck 44 of the second member and the intermediate member involve a first snap, wherein the respective covering films 30 and 50 of the first and second member are not torn by the blades 63 to avoid mixing, and a second snap wherein the respective covering films 30 and 50 of the first and second member are torn by the blades 63 to allow mixing.

Furthermore, two containers 2 and 4 of different length and substantially the same cross section have been illustrated in the figures. However, the containers 2 and 4 can have different dimensions from those illustrated; in particular, the second container 4, intended to receive the mixture, is advantageously wider than the container 2.

Furthermore, in the present description specific reference has been made to a powder substance 22 that is mixed with a liquid substance 42. However, the substances for mixing can be of any type, such as, for example, two powder substances, two viscous substances or two liquid substances.

Numerous variations or modifications of detail within the reach of a person skilled in the art can be made to the present embodiment of the invention, without thereby departing from the scope of the invention set forth in the appended claims.

1. A kit for mixing substances for the creation of cosmetic products, comprising:
a first end member containing a first substance and being closed at one end by a breakable cover;
a second end member containing a second substance and being closed at one end by a breakable cover; and
an intermediate member able to receive in an engagement relationship the ends provided with a breakable cover of said first and second member, said intermediate member comprising cutting means able to tear said breakable covers of said first and second member to allow discharge of the respect substances and defining a mixing chamber able to allow the passage of said substances for mixing thereof so as to obtain a read-to-use cosmetic product.

2. The kit of claim 1, wherein said first and second member comprise a substantially cylindrical body and a substantially cylindrical neck having a smaller diameter than the body and said breakable covers are disposed on the end rim of the neck.

3. The kit of claim 2, wherein said necks of said first and second member have engagement means able to sealably engage in said intermediate member.

4. The kit of claim 3, wherein said engagement means comprise at least one collar protruding outward form said necks of said first and second member.

5. The kit of claim 4, wherein said collar comprises an upper surface protruding obliquely from the side wall of the neck and a bottom surface protruding radially from the side wall of the neck.

6. The kit of claim 4, wherein said collar has at least one notch able to engage, in a sliding relationship, with a respective longitudinal projection provided in the inner side surface of said intermediate member.

7. The kit of claim 1, wherein said intermediate member comprises a substantially cylindrical body having a transverse bulkhead supporting a hollow cylinder in an axial position, defining said mixing chamber.

8. The kit of claim 7, wherein said cutting means provide two cusp-shape blades disposed respectively on the top and bottom rim of said cylinder of the intermediate member.

9. The kit of claim 7, wherein inside said intermediate member there are provided two longitudinal tongues supported by said transverse bulkhead so that each tongue has a flexible upper and lower part, each tongue being disposed between the inner surface of the intermediate member and the outer surface of the cylinder so that an annular gap able to receive said necks is created between the inner surface of the intermediate member and the outer surface of the cylinder, able to receive said necks of said first and second member.

10. The kit of claim 1, wherein said first member, said second member and said intermediate member are separate from each other and can be assembled at a time of use.

11. The kit of claim 1, wherein said first member and said second member are assembled to said intermediate member with their breakable covers kept apart from said cutting means and able to be brought into engagement therewith to cause rupture of said covers following a voluntary action by the user.

12. The kit of claim 1, wherein said first member and second member have different transverse and longitudinal sizes from one another.

13. The kit of claim 1, wherein a cosmetic powder product containing active principles of 1-ascorbic acid is contained inside said first member and a liquid is contained inside said second member.
